(19) United States
(12) Patent Application Publication
(10) Pub. No.: US 2019/0008721 A1
(43) Pub. Date: Jan. 10, 2019

(54) MULTIFUNCTIONAL FACIAL MASK AND ASSEMBLY THEREOF

(71) Applicant: Yuan Hsu, Taipei City (TW)

(72) Inventor: Yuan Hsu, Taipei City (TW)

(21) Appl. No.: 15/708,145

(22) Filed: Sep. 19, 2017

(30) Foreign Application Priority Data
Jul. 4, 2017 (TW) 106209807

(51) Int. Cl. A61H 39/00; A61N 1/32; A61N 5/06; A61N 1/04; A61N 1/02; A45D 44/00

(52) U.S. Cl. CPC A61H 39/002; A61N 1/328; A61N 5/0616; A61N 1/0476; A61N 1/025; A61H 2201/50; A61H 2039/005; A61H 2201/105; A61N 2005/0659; A61N 2005/0652; A61N 2005/0626; A45D 44/002

(57) ABSTRACT

Disclosed is a multifunctional facial mask, as well as an assembly thereof, which includes a mask body for attaching to a user's face and including first and conductor wires mounted thereto and spaced from each other. Thus, electrical power may be received through a facial cover to feed to the first and second conductor wires of the multifunctional facial mask to selectively conduct electronic massage on the user's face, so that in conducting caring of the user's face, an additional purpose of relaxing the user, physically and mentally, can be achieved.

### (A) TECHNICAL FIELD OF THE INVENTION

The present invention relates generally to a facial mask, and more particularly to a multifunctional facial mask featuring electronic massage and an assembly thereof.

### (B) DESCRIPTION OF THE PRIOR ART

To get good looking is the nature of human beings. People always work hard to pursue beautiful and good-looking appearance. However, due to the inevitable tendency of the living environment getting deteriorating, periodical use of facial masks is now prevailing among people who care more about beautifulness and good looking.

A great variety of facial mask products are currently available in the skin care market. Concerning functionality, there are moisture-keeping and nutrition-supplementing masks, cleaning masks, acne-caring masks, and beauty and whitening masks. For those concerned about beauty and good looking, although the function and being easy to use achieved with the known masks are satisfactory. However, in the modern society, people are living under tremendous pressures that often cause all sorts of sickness and tiredness. It is thus desired to make improvements on the facial masks that are commonly used to provide, in addition to caring of facial skin, a solution for relaxing the user physically, mentally, and spiritually, as an advantageous option for the user.

### SUMMARY OF THE INVENTION

In view of the above, an objective of the present invention is to provide a multifunctional facial mask and an assembly thereof, which allows for relaxation of a user, both physically and mentally, by applying electronic massage to the face of the user, while being used for caring the face of the user.

To achieve the above and other objectives, the present invention provides a multifunctional facial mask and a multifunctional facial mask assembly that additionally includes a facial cover. The multifunctional facial mask comprises a mask body for attaching to a user's face and a first conductor wire and a second conductor wire that are mounted to the mask body and are spaced from each other. Thus, electrical power is received through the facial cover to feed to the first conductor wire and the second conductor wire of the multifunctional facial mask to selectively conduct electronic massage on the user's face.

In an embodiment, the first conductor wire and the second conductor wire of the multifunctional facial mask are formed of conductive silicon rubber or conductive paint and are arranged to respectively extend from locations on the facial mask corresponding to two zanzhu acupoints of the face of the user.

In an embodiment, the first conductor wire and the second conductor wire of the multifunctional facial mask respectively extend from locations on the facial mask that correspond to the two zanzhu acupoints of the face of the user in leftward and rightward directions and are further extended in upward and downward directions as two curved lines along a contour of the face of the user, the two curved lines being connected to a plurality of horizontal lines extended from the curved lines in horizontal directions and alternate with each other.

In an embodiment, the first conductor wire and the second conductor wire of the multifunctional facial mask respectively extend from locations on the facial mask that correspond to two zanzhu acupoints of the face of the user in upward and downward directions and are further extended in leftward and rightward directions to be each in the form of multiple substantially circular arc lines that are alternate with each other.

In an embodiment, the mask body of the multifunctional facial mask is selected as one of cotton fabric, knitted fabric, and non-woven fabric.

In an embodiment, the mask body of the multifunctional facial mask comprises a material that emits far infrared light mounted thereto.

In an embodiment, the mask body of the multifunctional facial mask is filled with essence liquid that is good for facial skin.

In an embodiment, the mask body of the multifunctional facial mask has a surface that is printed with a pattern for outside aesthetics.

In an embodiment, the facial cover of the multifunctional facial mask assembly comprises a cover body and an electrical power controller. The cover body comprises at least two the electrodes respectively corresponding to the first conductor wire and the second conductor wire and the electrical power controller is connected to the electrodes of the cover body to control type and magnitude of the electrical power supplied through the first conductor wire and the second conductor wire.

In an embodiment, the cover body of the multifunctional facial mask assembly further comprises a plurality of light-emitting diodes mounted thereto and connected to the electrical power controller.

In an embodiment, the cover body of the multifunctional facial mask assembly further comprises a plurality of Hokutolite stones mounted thereto to provide a therapeutic effect.

In an embodiment, the cover body of the multifunctional facial mask assembly is made of an acrylic material or a plastic material of acrylonitrile butadiene styrene (ABS).

In summary, the multifunctional facial mask and the assembly thereof, in addition to the mask body for attaching to a user's face, further comprise the first conductor wire and the second conductor wire that are mounted to the mask body in a manner of being spaced from each other. Thus, electrical power can be received through the facial cover to feed to the first conductor wire and the second conductor wire of the multifunctional facial mask for selectively conducting electronic massage on the user's face, so that in conducting caring of the user's face, an additional purpose of relaxing the user, physically and mentally, can be achieved.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a schematic view showing a multifunctional facial mask according to a first embodiment of the present invention.

FIG. 2 is a schematic view showing a multifunctional facial mask according to a second embodiment of the present invention.

FIG. 3 is a schematic view illustrating a facial cover as an assembly with a multifunctional facial mask according to a preferred embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

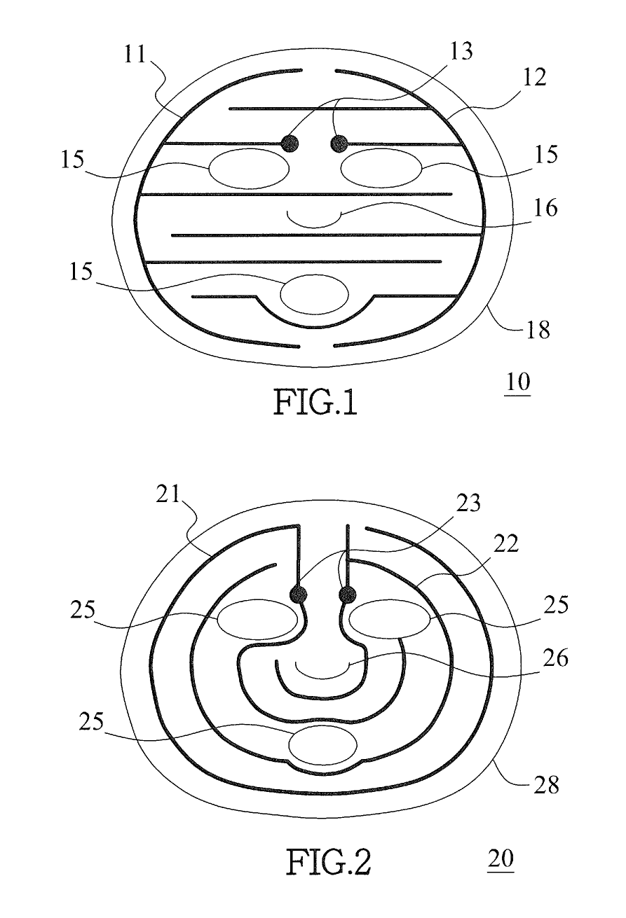

Referring to FIG. 1, a schematic view is given to show a multifunctional facial mask according to a first embodiment of the present invention. In the drawing, the multifunctional facial mask 10 is shown comprising a mask body 18 for attaching to a user's face and also comprising a first conductor wire 11 and a second conductor wire 12 that are mounted to the mask body 18 and are spaced from each other so that the first conductor wire 11 and the second conductor wire 12 may conduct in for example electrical power in the form of a direct current or an alternate current and selectively adjustable for energy intensity carried thereby to selectively carry out electronic massage on the user's face.

In this embodiment, the first conductor wire 11 and the second conductor wire 12 can be for example electrically conductive silicon rubber or electrically conductive paint that are made with a printing operation. The mask body 18 is formed of for example a material selected from one of cotton fabric, knitted fabric, or non-woven fabric. The mask body 18 is formed with a plurality of openings 15 and/or cut slits 16 corresponding to the eyes, the mouth, and the nose of the user to allow for easy application to the user's face. The mask body 18 can be selectively impregnated with or coated with essence liquid that is good for the facial skin, or can be embedded therein with a material that emits far infrared light. The mask body 18 has a surface that may be selectively printed with a pattern for outside aesthetic decoration to improve the quality of the multifunctional facial mask 10.

The first conductor wire 11 and the second conductor wire 12 respectively extend from locations 13 of the mask body that correspond to two zanzhu acupoints (around inner ends of the eyebrows) of the user's face in leftward and rightward direction and then further extend, as two curved lines along the contour of the user's face, in both upward and downward directions, and the two curved lines are further connected to a plurality of horizontal lines that are extended from the curved lines generally in horizontal directions and are arranged alternate with each other so that such a distributive arrangement of the first conductor wire 11 and the second conductor wire 12 allows for massage applied to multiple acupoints of the user's face, including the two zanzhu acupoints to thereby help release or alleviate for example migraine headache, all sorts of headaches or head pains, or sleep disorder and thus releasing the stress of the user.

Referring to FIG. 2, a schematic view is given to illustrate a multifunctional facial mask according to a second embodiment of the present invention. Similar to the first embodiment, the second embodiment provides a multifunctional facial mask 20 that, in addition to a mask body 28 that is attachable to the user's face, comprises a first conductor wire 21 and a second conductor wire 22 that are mounted to the mask body 28 in a manner of being spaced from each other.

In this embodiment, the first conductor wire 21 and the second conductor wire 22 can be for example electrically conductive silicon rubber or electrically conductive paint that are made with a printing operation. The mask body 28 is formed of for example a material selected from one of cotton fabric, knitted fabric, or non-woven fabric. The mask body 28 is similarly formed with a plurality of openings 25 and/or cut slits 26 corresponding to the eyes, the mouth, and the nose of the user to allow for easy application to the user's face. The mask body 28 can also be selectively impregnated with or coated with essence liquid that is good for the facial skin, or can be embedded therein with a material that emits far infrared light. The mask body 28 has a surface that may be selectively printed with a pattern for outside aesthetic decoration to improve the quality of the multifunctional facial mask 20.

What is different from the first embodiment is that the first conductor wire 21 and the second conductor wire 22 of the multifunctional facial mask 20 are arranged to respectively extend from locations 23 of the mask body that correspond to two zanzhu acupoints (around inner ends of the eyebrows) of the user's face in upward and downward direction and are further extended in leftward and rightward direction, each in the form of multiple roughly circular arc lines that are alternate with each other so that such a distributive arrangement of the first conductor wire 21 and the second conductor wire 22 allows for massage applied to multiple acupoints of the user's face, including the two zanzhu acupoints to thereby help release or alleviate for example migraine headache, all sorts of headaches or head pains, or sleep disorder and thus releasing the stress of the user.

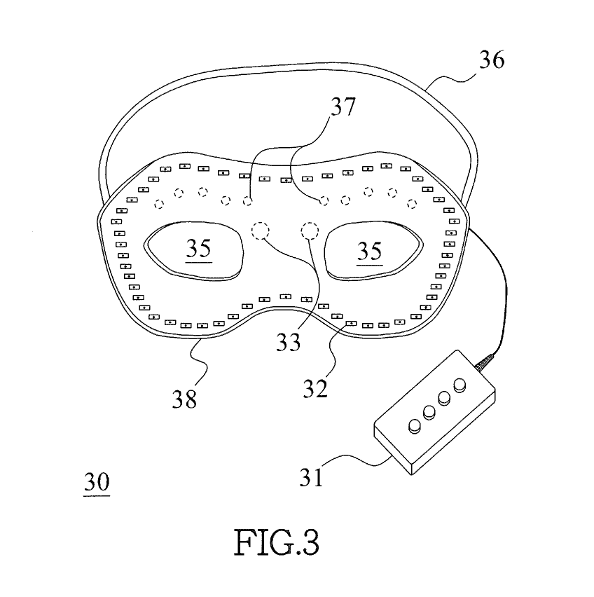

Referring to FIG. 3, a schematic view is given to illustrate a facial cover as an assembly with a multifunctional facial mask according to a preferred embodiment of the present invention. The facial cover 30 may be used in selectively combination with the multifunctional facial mask 10 of FIG. 1 or the multifunctional facial mask 20 of FIG. 2. In the drawing, the facial cover 30 comprises a cover body 38 and an electrical power controller 31. The cover body 38 is of a three-dimensional configuration that generally matches the contour of the user's face and is made of a material that is selected from an acrylic material that shows slight elasticity or a plastic material of acrylonitrile butadiene styrene (ABS) to provide comfortableness of wear.

The cover body 38 is selectively provided with an elastic strip 36 attached thereto for retaining the facial cover 30 on the user's face and is selectively provided with openings 35 at suitable locations thereof to respectively correspond to the user's eyes, and is further provided, on a circumferential marginal portion thereof, a plurality of light-emitting diodes 32 connected to the electrical power controller 31. The cover body 38 may further comprise a plurality of Hokutolite stones 37 embedded therein or mounted thereto to provide a therapeutic effect and also comprises at least two electrodes 33 mounted to a surface thereto to correspond to the first conductor wire 11, 21 and the second conductor wire 12, 22 of the multifunctional facial mask 10, 20. The electrodes 33 are connected to the electrical power controller 31 of the facial cover 30 to conduct electrical power from the electrical power controller 31 to the first conductor wire 11, 21 and the second conductor wire 12, 22 of the multifunctional facial mask 10, 20 to selectively carry out electronic massage on the user's face.

In this embodiment, the electrical power from the electrical power controller 31 can be for example an electrical battery or a secondary cell that is arranged in a housing and supplies a direct current or an alternate current from an external household power supply. The electrical power fed from the electrical power controller 31 to the electrodes 33 can be a direct current or an alternate current, of which a frequency or magnitude is selected by a user by operating the electrical power controller 31.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A multifunctional facial mask, comprising:
a mask body, which is adapted to attach to a face of a user;
a first conductor wire, which is mounted to the mask body; and
a second conductor wire, which is mounted to the mask body and spaced from the first conductor wire so that when electrical power is supplied through the first conductor wire and the second conductor wire, electronic massage is selectively conducted on the face of the user.

2. The multifunctional facial mask according to claim 1, wherein the first conductor wire and the second conductor wire are formed of conductive silicon rubber or conductive paint and are arranged to respectively extend from locations on the facial mask corresponding to two zanzhu acupoints of the face of the user.

3. The multifunctional facial mask according to claim 1, wherein the first conductor wire and the second conductor wire respectively extend from locations on the facial mask that correspond to the two zanzhu acupoints of the face of the user in leftward and rightward directions and are further extended in upward and downward directions as two curved lines along a contour of the face of the user, the two curved lines being connected to a plurality of horizontal lines extended from the curved lines in horizontal directions and alternate with each other.

4. The multifunctional facial mask according to claim 1, wherein the first conductor wire and the second conductor wire respectively extend from locations on the facial mask that correspond to two zanzhu acupoints of the face of the user in upward and downward directions and are further extended in leftward and rightward directions to be each in the form of multiple substantially circular arc lines that are alternate with each other.

5. The multifunctional facial mask according to claim 1, wherein the mask body is selected as one of cotton fabric, knitted fabric, and non-woven fabric.

6. The multifunctional facial mask according to claim 1, wherein the mask body comprises a material that emits far infrared light mounted thereto.

7. The multifunctional facial mask according to claim 1, wherein the mask body is filled with essence liquid that is good for facial skin.

8. The multifunctional facial mask according to claim 1, wherein the mask body has a surface that is printed with a pattern for outside aesthetics.

9. A multifunctional facial mask assembly, comprising:
a mask body, which is adapted to attach to a face of a user;
a first conductor wire, which is mounted to the mask body;
a second conductor wire, which is mounted to the mask body and spaced from the first conductor wire; and
a facial cover, which receives electrical power through the first conductor wire and the second conductor wire to conduct selectively electronic massage on the face of the user.

10. The multifunctional facial mask assembly according to claim 9, wherein the first conductor wire and the second conductor wire are formed of conductive silicon rubber or conductive paint and are arranged to respectively extend from locations on the facial mask corresponding to two zanzhu acupoints of the face of the user.

11. The multifunctional facial mask assembly according to claim 9, wherein the first conductor wire and the second conductor wire respectively extend from locations on the facial mask that correspond to the two zanzhu acupoints of the face of the user in leftward and rightward directions and are further extended in upward and downward directions as two curved lines along a contour of the face of the user, the two curved lines being connected to a plurality of horizontal lines extended from the curved lines in horizontal directions and alternate with each other.

12. The multifunctional facial mask assembly according to claim 9, wherein the first conductor wire and the second conductor wire respectively extend from locations on the facial mask that correspond to two zanzhu acupoints of the face of the user in upward and downward directions and are further extended in leftward and rightward directions to be each in the form of multiple substantially circular arc lines that are alternate with each other.

13. The multifunctional facial mask assembly according to claim 9, wherein the mask body is selected as one of cotton fabric, knitted fabric, and non-woven fabric.

14. The multifunctional facial mask assembly according to claim 9, wherein the mask body comprises a material that emits far infrared light mounted thereto.

15. The multifunctional facial mask assembly according to claim 9, wherein the mask body is filled with essence liquid that is good for facial skin.

16. The multifunctional facial mask assembly according to claim 9, wherein the mask body has a surface that is printed with a pattern for outside aesthetics.

17. The multifunctional facial mask assembly according to claim 9, wherein the facial cover comprises:
a cover body, which comprises at least two the electrodes respectively corresponding to the first conductor wire and the second conductor wire; and
an electrical power controller, which is connected to the electrodes to control type and magnitude of the electrical power supplied through the first conductor wire and the second conductor wire.

18. The multifunctional facial mask assembly according to claim 17, wherein the cover body comprises a plurality of light-emitting diodes mounted thereto and connected to the electrical power controller.

19. The multifunctional facial mask assembly according to claim 17, wherein the cover body comprises a plurality of Hokutolite stones mounted thereto.

20. The multifunctional facial mask assembly according to claim 17, wherein the cover body is made of an acrylic material or a plastic material of acrylonitrile butadiene styrene (ABS).
